(19) United States
(12) Patent Application Publication
(10) Pub. No.: US 2012/0172289 A1
(43) Pub. Date: Jul. 5, 2012

(54) MULTIFUNCTIONAL GLYCOPEPTIDE ANTIBIOTIC DERIVATIVES FOR FLUORESCENT IMAGING AND PHOTOACTIVE ANTIMICROBIAL THERAPY

(75) Inventors: Bengang Xing, Singapore (SG); Tingting Jiang, Singapore (SG); Roushen Liew, Singapore (SG)

(73) Assignee: NANYANG TECHNOLOGICAL UNIVERSITY, Singapore (SG)

(21) Appl. No.: 13/339,081

(22) Filed: Dec. 28, 2011

Related U.S. Application Data
(60) Provisional application No. 61/428,502, filed on Dec. 30, 2010.

(51) Int. Cl. A61K 38/14; C07D 487/22; A61K 31/409; A61N 5/06; G01N 21/64; C07K 1/04; A61P 31/04; C07K 17/02; G01N 21/62

(52) U.S. Cl. 514/2.6; 530/322; 540/145; 514/20.9; 514/410; 514/2.9; 435/34; 607/88; 514/2.7

(57) ABSTRACT

The present invention relates to a compound of formula (I) and compositions thereof, methods of their production as well as methods for treating bacterial infection.

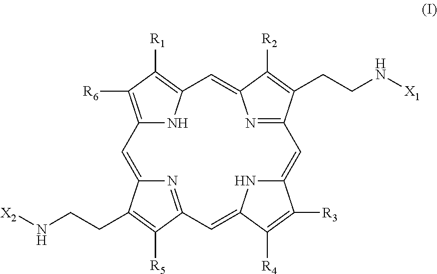

### CROSS REFERENCE TO RELATED APPLICATIONS

This application makes reference to and claims the benefit of priority of an application for “Multifunctional glycopeptides antibiotic derivatives for fluorescent imaging and photoactive antimicrobial therapy” filed on Dec. 30, 2010 with the United States Patent and Trademark Office, and there duly assigned serial number U.S. Provisional 61/428,502. The contents of said application filed on Dec. 30, 2010 is incorporated herein by reference for all purposes, including an incorporation of any element or part of the description, claims or drawings not contained herein.

### FIELD OF THE INVENTION

The present invention relates to glycopeptide antibiotic derivatives and compositions thereof, methods of their production as well as methods for treating bacterial infection.

### BACKGROUND OF THE INVENTION

Vancomycin (Van) is a powerful glycopeptide antibiotic to treat methicillin-resistance Gram-positive infections through their specific binding affinity to the C-terminal L-Lys-D-Ala-D-Ala motif present in bacterial cell wall precursors.¹ However, bacteria having resistance to vancomycin known as vancomycin-resistant enterococci (VRE) recently emerged as a serious threat to public health, which is typically due to the mutation of peptidoglycan sequence from D-Ala-D-Ala to D-Ala-D-Lac, resulting in the substantial decrease of binding affinity (˜10³ times loss) to the Van molecule.¹ Extensive studies done by Griffin,² Nicolaou,³ Williams⁴ and Whitesides et al⁵ revealed that covalently linked dimers and oligomers of Van could serve as promising approaches to enhance the potent activities against VRE based on the polyvalent/multivalent interactions to circumvent the low affinities binding between Van and D-Ala-D-Lac peptide precursors in resistant bacteria.⁶ However, recent reports also indicated that increased binding affinity may not always lead to substantial activities with effective minimum inhibitory concentration (MIC) against VRE organisms.^{4,7}

One promising alternative for microbiological control is based on photodynamic antimicrobial chemotherapy (PACT),⁸ which involves the use of photosensitizers to generate reactive oxygen species (ROS, e.g. singlet oxygen (¹O₂)) upon light exposure at a suitable wavelength. These reactive oxygen species are cytotoxic and are capable of destroying the cell walls and membranes, thus resulting in cell death.⁹ To date, PACT has been demonstrated to be effective against a variety of Gram-positive and Gram-negative bacteria.^{8,9} One possibility to minimize side effects and further improve the efficiency of PACT in clinics is the use of affinity ligands that can efficiently target photosensitizers to areas of bacterial infections. Several affinity ligands based on antibodies,¹⁰ protein cage,¹¹ polypeptide,¹² nanoparticles,¹³ and bacteriophage¹⁴ have been reported to successfully direct lethal photosensitizers to antibiotic-resistant bacteria. However, there are still drawbacks for these affinity groups to target bacterial surface. Therefore, there remains a need to develop simpler and economical targeting molecules capable of overcoming the above problems, since most of current approaches are complicated, require tedious manipulation and may suffer from difficulty in synthesis, self-aggregation or possible immunogenicity.¹⁰⁻¹⁴

### SUMMARY OF THE INVENTION

In a first aspect, the present invention relates to a compound of formula (I)

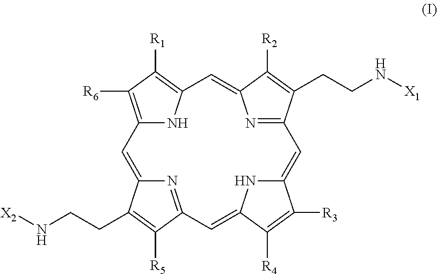

wherein

R₁ to R₆ are each independently selected from the group consisting of unsubstituted or substituted C₁-C₁₀ alkyl, unsubstituted or substituted C₁-C₁₀ alkenyl, unsubstituted or substituted C₁-C₁₀ alkynyl and unsubstituted or substituted C₁-C₁₀ alkoxy;

X₁ and X₂ are each independently H or vancomycin;

or a tautomer, stereoisomer, pharmaceutically acceptable salt or prodrug thereof.

In a second aspect, the present invention relates to a composition comprising a compound of the present invention.

In a third aspect, the present invention relates to a method of treating a bacterial infection in a subject. The method includes administering a therapeutically effective amount of the compound or the composition of the present invention to a subject in need thereof.

In a fourth aspect, the present invention relates to a method of detecting a bacterium. The method includes contacting said bacterium with at least one compound of the present invention, wherein the bacterium is detected by detecting the binding between the compound and the said bacterium.

In a fifth aspect, the present invention relates to a method of preparing a compound of formula (I). The method includes reacting vancomycin hydrochloride with a compound of formula (IV)

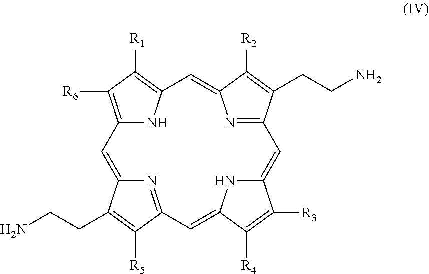

in the presence of a coupling reagent, under conditions to form a compound of formula (I), wherein

R₁ to R₆ are each independently selected from the group consisting of unsubstituted or substituted C₁-C₁₀ alkyl, unsubstituted or substituted C₁-C₁₀ alkenyl, unsubstituted or substituted C₁-C₁₀ alkynyl and unsubstituted or substituted C₁-C₁₀ alkoxy.

### BRIEF DESCRIPTION OF THE DRAWINGS

In order to better understand the present invention and to demonstrate how it may be carried out in practice, illustrative embodiments will now be described by way of non-limiting examples only, with reference to the accompanying drawings, in which

FIG. 1 shows the interaction between a compound according to one embodiment of the present invention and Gram positive bacterial cell wall.

FIG. 2 illustrates the synthetic pathway for preparing a compound according to one embodiment of the present invention. The commercially available vancomycin (Van) (“1”) reacted with a porphyrin derivative (“2”), to afford Van carboxamide (“3b”) by employing O-benzotriazol-1 -yl-N,N,N′,N′ -tetramethyl-uronium-hexafluorophosphate (HBTU) as the coupling reagent. The divalent conjugate (compound of Formula II) was purified in 53.6% yield by reversed-phase HPLC and characterized by ¹H NMR spectroscopy and matrix-assisted laser desorption ionization time-of-flight mass spectrometry (MALDI-TOF-MS). Similarly, the monovalent Van adduct (“3a”) with porphyrin was also prepared in 52.9% yield by using excess amount of porphyrin.

FIG. 3 shows the photochemical properties of the compounds of the present invention for example the compounds of formula II and III (see compounds “3b” and “3a”) as compared to the porphyrin derivative (“2”), according to various embodiments of the invention. The absorption (a) and fluorescence (b) spectra of vancomycin (“1”), porphyrin derivative (“2”), compound of formula II (“3b”) and compound of formula III (“3a”) in PBS (1% DMSO) at room temperature (λₑₓ=530 nm). The UV-visible and fluorescence spectroscopy results indicated that the compounds of the invention exhibited absorption bands of both vancomycin (˜280 nm) and porphyrin moieties (B band around 400 nm, Q bands between 500 and 620 nm). The emission spectra of the compounds of formula II and III (compounds “3b” and “3a”) showed no difference from those of the porphyrin derivative (“2”), thereby indicating that vancomycin conjugations have no effect on the fluorescent property of porphyrin.

FIG. 4 shows the fluorescent intensity of 9,10-anthracenediyl-bis(methylene)dimalonic acid (ABDA) (20 μM) mixed with compounds 2 (porphyrin derivative), 3a (compounds of formula III) and 3b (compound of formula II) (10 μM) in PBS buffer before and after light irradiation for 2 min. The destruction of ABDA indicated the generation of singlet oxygen. Excitation: 380 nm; Emission: 431 nm.

FIG. 5 shows the fluorescent imaging of bacterial staining with the compounds of the invention. (a)-(c), B. subtilis loaded with 2 μM of 3b (compound of formula II), 3a (compound of formula III), and 2 (porphyrin derivative), respectively; (d)-(f), E. faecium (VanA) with 10 μM of 3b, 3a, and 2; (g)-(i), E. faecalis (VanB) with 10 μM of 3b, 3a, and 2. Ex=535/50 nm; Em=610/75 nm.

FIG. 6 shows the fluorescent imaging of bacterial staining with the compounds of the present invention. (a)-(c), B. subtilis loaded with 2 μM of 3b (compound of formula II), 3a (compound of formula III), and 2 (porphyrin derivative), respectively; (d)-(f), E. faecium (VanA) loaded with 2 μM of 3b, 3a, and 2; (g) - (i), E. faecalis (VanB) loaded with 2 μM of 3b, 3a, and 2. Ex=535/50 nm; Em=610/75 nm.

FIG. 7 shows the differential interference contrast images and fluorescent images of bacterial staining. (a)-(f) Stained cells of E. Faecium; (g)-(i) Stained cells of E. Faecalis; Left: compound of formula II (“3b”); Middle: compound of formula III (“3a”); right: Porphyrin (“2”); The concentration of all species was 10 μM (λx=535/50 nm; λem=610/75 nm).

FIG. 8 shows the photodynamic inactivation of bacterial strains towards different concentration of compounds 2 (porphyrin derivative), 3a (compound of formula III) and 3b (compound of formula II). (a): E. faecium (VanA); (b): E. faecalis (VanB). The white-light dose was 60 J/cm² (exposure for 2 min at a fluence rate of 500 mW/cm²). Bacteria treated with compound 3b but no light illumination as control groups.

FIG. 9 show the light dose-dependent bacterial lethality towards different compounds 2 (porphyrin derivative), 3a (compound of formula III) and 3b (compound of formula II). (a): E. faecium (VanA); (b): E. faecalis (VanB). The concentration of all compounds was 2 μM. Bacteria treated with light illumination (without any photosensitizers) as control groups.

FIG. 10 shows the photodynamic antibacterial activity towards B. subtilis in the presence of compounds 2 (porphyrin derivative), 3a (compound of formula III) and 3b (compound of formula II). (a) bacterial lethality with incubation of different concentration of compounds 2, 3a and 3b in the dark. (b) bacterial lethality with incubation of different concentration of compounds 2, 3a and 3b upon 60 J/cm² of white light illumination. (c) Light dose-dependent bacterial lethality with incubation of 0.5 μM 2, 3a and 3b in the presence of different doses of white light irradiation. B. subtilis treated with light illumination without any compound incubation was used as control group.

FIG. 11 shows the photodynamic antibacterial activity towards E. coli in the presence of compounds 2 (porphyrin derivative), 3a (compound of formula III) and 3b (compound of formula II). (a) Bacterial lethality with incubation of different concentration of compounds 2, 3a and 3b. The white-light dose was 60 J/cm². Bacteria treated with compound 3b but no light illumination as control groups. (b) Light dose-dependent bacterial lethality with incubation of 2 μM 2, 3a and 3b in the presence of different doses of white light irradiation. E. coli treated with light illumination without any compound incubation was used as control group.

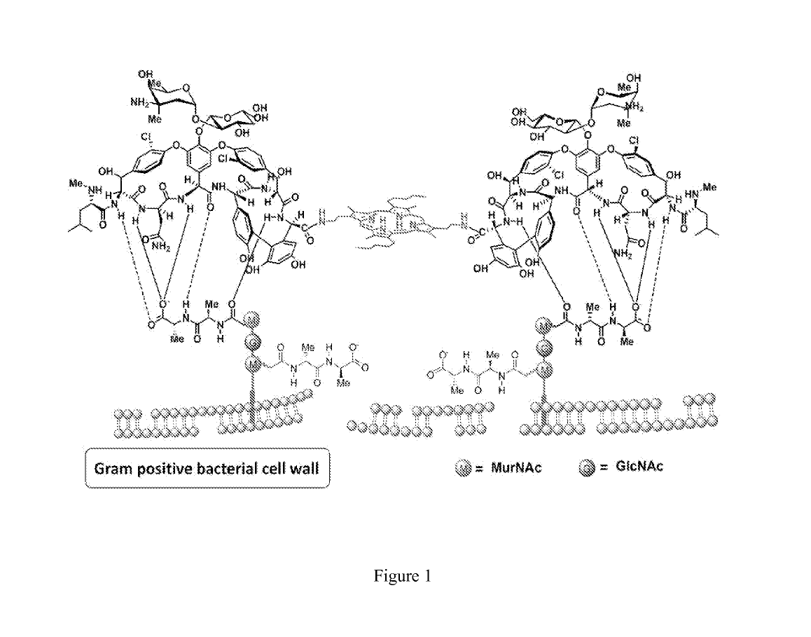

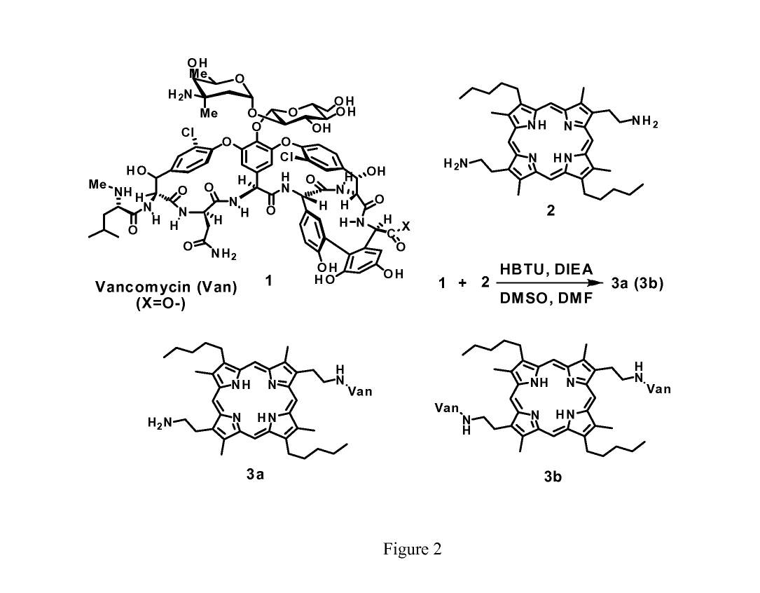

### DETAILED DESCRIPTION OF THE INVENTION

### Definitions

Unless otherwise stated the following terms used in the specification and claims have the meanings discussed below:

An “alkyl” refers to a saturated aliphatic hydrocarbon including straight chain, or branched chain groups. Preferably, the alkyl group has 1 to 10 carbon atoms (whenever a numerical range; e.g.,“1-10”, is stated herein, it means that the group, in this case the alkyl group, may contain 1 carbon atom, 2 carbon atoms, 3 carbon atoms, etc. up to and including 10 carbon atoms). More specifically, it may be a medium size alkyl having 1 to 6 carbon atoms or a lower alkyl having 1 to 4 carbon atoms e. g., methyl, ethyl, n-propyl, isopropyl, butyl, iso-butyl, tert-butyl, pentyl, n-pentyl and the like. The alkyl group may be substituted or unsubstituted. When substituted, the substituent group(s) is one or more, for example one two, three, four or five groups, individually selected from the group consisting of C₃-C₈ cycloalkyl, C₆-C₁₄ aryl, 5-10 membered heteroaryl wherein 1 to 4 ring atoms are independently selected from nitrogen, oxygen or sulfur, 5-10 membered heteroalicyclic wherein 1 to 3 ring atoms are independently nitrogen, oxygen or sulfur, hydroxy, C₁-C₁₀ alkoxy, C₃-C₈ cycloalkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, C-carboxy, O-carboxy, nitro, silyl, sulfinyl, sulfonyl, amino, and —NR¹⁰R¹¹ where R¹⁰ and R¹¹ are independently selected from the group consisting of hydrogen, C₁-C₄ alkyl, C₃-C₈ cycloalkyl, C₆-C₁₄ aryl, carbonyl, acetyl, sulfonyl, amino, and trifluoromethanesulfonyl, or R¹⁰ and R¹¹, together with the nitrogen atom to which they are attached, combine to form a five-or six-membered heteroalicyclic ring.

An “alkenyl” group refers to an alkyl group, as defined herein, consisting of at least two carbon atoms and at least one carbon-carbon double bond e. g., ethenyl, propenyl, butenyl or pentenyl and their structural isomeric forms such as 1-or 2-propenyl, 1-, 2-, or 3-butenyl and the like. The alkenyl group may be substituted or unsubstituted. When substituted, the substituent group(s) can be one or more of the substituent group(s) defined above.

An “alkynyl” group refers to an alkyl group, as defined herein, consisting of at least two carbon atoms and at least one carbon-carbon triple bond e. g., acetylene, ethynyl, propynyl, butynyl, or pentynyl and their structural isomeric forms as described above. The alkynl group may be substituted or unsubstituted. When substituted, the substituent group(s) can be one or more of the substituent group(s) defined above.

An “alkoxy” group refers to an —O-unsubstituted alkyl and —O-substituted alkyl group, as defined herein. Examples include and are not limited to methoxy, ethoxy, propoxy, butoxy, and the like. The alkoxy group may be substituted or unsubstituted. When substituted, the substituent group(s) can be one or more of the substituent group(s) defined above.

An “aryl” group refers to an all-carbon monocyclic or fused-ring polycyclic (i.e., rings which share adjacent pairs of carbon atoms) groups of 6 to 14 ring atoms and having a completely conjugated pi-electron system. Examples, without limitation, of aryl groups are phenyl, naphthalenyl and anthracenyl. The aryl group may be substituted or unsubstituted. When substituted, the substituted group(s) is one or more, for example one, two, or three substituents, independently selected from the group consisting of C₁-C₁₀ alkyl, C₃-C₈ cycloalkyl, C₆-C₁₄ aryl, 5-10 membered heteroaryl wherein 1 to 4 ring atoms are independently selected from nitrogen, oxygen or sulfur, 5-10 membered heteroalicyclic wherein 1 to 3 ring atoms are independently nitrogen, oxygen or sulfur, hydroxy, C₁-C₁₀ alkoxy, C₃-C₈ cycloalkoxy, aryloxy, mercapto, alkylthio, arylthio, cyano, halo, trihalomethyl, carbonyl, thiocarbonyl, O-carbamyl, N-carbamyl, O-thiocarbamyl, N-thiocarbamyl, C-amido, N-amido, C-carboxy, O-carboxy, nitro, silyl, sulfinyl, sulfonyl, amino, and —NR¹⁰R¹¹, with R¹⁰ and R¹¹ as defined above. Preferably the substituent(s) is/are independently selected from chloro, fluoro, bromo, methyl, ethyl, hydroxy, methoxy, nitro, carboxy, methoxycarbonyl, sulfonyl, or amino.

A “heteroaryl” group refers to a monocyclic or fused aromatic ring (i.e., rings which share an adjacent pair of atoms) of 5 to 10 ring atoms in which one, two, three or four ring atoms are selected from the group consisting of nitrogen, oxygen and sulfur and the rest being carbon. Examples, without limitation, of heteroaryl groups are pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, isobenzofuryl, benzothienyl, benzotriazolyl, isobenzothienyl, indolyl, isoindolyl, 3H-indolyl, benzimidazolyl, benzothiazolyl, benzoxazolyl, quinolizinyl, quinazolinyl, pthalazinyl, quinoxalinyl, cinnnolinyl, napthyridinyl, quinolyl, isoquinolyl, tetrazolyl, 5,6,7,8-tetrahydroquinolyl, 5,6,7,8-tetra-hydroisoquinolyl, purinyl, pteridinyl, pyridinyl, pyrimidinyl, carbazolyl, xanthenyl or benzoquinolyl. The heteroaryl group may be substituted or unsubstituted. When substituted, the substituted group(s) is one or more, for example one or two substituents, independently selected from the group consisting of C₁-C₁₀ alkyl, C₃-C₈ cycloalkyl, C₆-C₁₄ aryl, 5-10 membered heteroaryl wherein 1 to 4 ring atoms are independently selected from nitrogen, oxygen or sulfur, 5-10 membered heteroalicyclic wherein 1 to 3 ring atoms are independently nitrogen, oxygen or sulfur, hydroxy, C₁-C₁₀ alkoxy, C₃-C₈ cycloalkoxy, aryloxy, mercapto, alkylthio, arylthio, cyano, halo, trihalomethyl, carbonyl, thiocarbonyl, O-carbamyl, N-carbamyl, O-thiocarbamyl, N-thiocarbamyl, C-amido, N-amido, C-carboxy, O-carboxy, nitro, silyl, sulfinyl, sulfonyl, amino, and —NR¹⁰R¹¹, with R¹⁰ and R¹¹ as defined above. Preferably the substituent(s) is/are independently selected from chloro, fluoro, bromo, methyl, ethyl, hydroxy, methoxy, nitro, carboxy, methoxycarbonyl, sulfonyl, or amino.

A “heteroalicyclic” group refers to a monocyclic or fused ring of 5 to 10 ring atoms containing one, two, or three heteroatoms in the ring which are selected from the group consisting of nitrogen, oxygen and —S(O)ₙ where n is 0-2, the remaining ring atoms being carbon. The rings may also have one or more double bonds. However, the rings do not have a completely conjugated pi-electron system. Examples, without limitation, of heteroalicyclic groups are pyrrolidine, piperidine, piperazine, morpholine, imidazolidine,tetrahydropyridazine, tetrahydrofuran, thiomorpholine, tetrahydropyridine, and the like. The heteroalicyclic ring may be substituted or unsubstituted. When substituted, the substituted group (s) is one or more, for example one, two, or three substituents, independently selected from the group consisting of C₁-C₁₀ alkyl, C₃-C₈ cycloalkyl, C₆-C₁₄ aryl, 5-10 membered heteroaryl wherein 1 to 4 ring atoms are independently selected from nitrogen, oxygen or sulfur, 5-10 membered heteroalicyclic wherein 1 to 3 ring atoms are independently nitrogen, oxygen or sulfur, hydroxy, C₁-C₁₀ alkoxy, C₃-C₈ cycloalkoxy, aryloxy, mercapto, alkylthio, arylthio, cyano, halo, trihalomethyl, carbonyl, thiocarbonyl, O-carbamyl, N-carbamyl, O-thiocarbamyl, N-thiocarbamyl, C-amido, N-amido, C-carboxy, O-carboxy, nitro, silyl, sulfinyl, sulfonyl, amino, and —NR¹⁰R¹¹, with R¹⁰ and R¹¹ as defined above. The substituent(s) is/are for example independently selected from chloro, fluoro, bromo, methyl, ethyl, hydroxy, methoxy, nitro, carboxy, methoxycarbonyl, sulfonyl, or amino.

A “hydroxy” group refers to an —OH group.

An “alkoxy” group refers to an —O-unsubstituted alkyl and —O-substituted alkyl group, as defined herein. Examples include and are not limited to methoxy, ethoxy, propoxy, butoxy, and the like.

A “cycloalkoxy” group refers to an —O-cycloalkyl group, as defined herein. One example is cyclopropyloxy.

An “aryloxy” group refers to both an —O-aryl and an —O-heteroaryl group, as defined herein. Examples include and are not limited to phenoxy, napthyloxy, pyridyloxy, furanyloxy, and the like.

A “mercapto” group refers to an —SH group.

An “alkylthio” group refers to both an S-alkyl and an —S-cycloalkyl group, as defined herein. Examples include and are not limited to methylthio, ethylthio, and the like.

An “arylthio” group refers to both an —S-aryl and an —S-heteroaryl group, as defined herein. Examples include and are not limited to phenylthio, napthylthio, pyridylthio, furanylthio, and the like.

A “halo” or “halogen” group refers to fluorine, chlorine, bromine or iodine.

A “cyano” group refers to a —CN group.

A “carbonyl” refers to a —C(═O)—R″ group, where R″ is selected from the group consisting of hydrogen, alkyl, cycloalkyl, aryl, heteroaryl (bonded through a ring carbon) and heteroalicyclic (bonded through a ring carbon), as defined herein. Representative examples include and the not limited to acetyl, propionyl, benzoyl, formyl, cyclopropylcarbonyl, pyridinylcarbonyl, pyrrolidin-lylcarbonyl, and the like.

A “thiocarbonyl” group refers to a —C(═S)—R″ group, with R″ as defined herein.

An “O-carbamyl” group refers to a —OC(═O)NR¹⁰R¹¹ group with R¹⁰ and R¹¹ as defined herein.

An “N-carbamyl” group refers to a R¹¹OC(═O) NR¹⁰— group, with R¹⁰ and R¹¹ as defined herein.

An “O-thiocarbamyl” group refers to a —OC(═S)NR¹⁰R¹¹ group, with R¹⁰ and

R¹¹ as defined herein.

An “N-thiocarbamyl” group refers to a R¹¹OC(═S)NR¹⁰— group, with R¹⁰ and R¹¹ as defined herein.

An “amino” group refers to an —NR¹⁰R¹¹ group, wherein R¹⁰ and R¹¹ are independently hydrogen or unsubstituted lower alkyl, e.g, —NH₂, dimethylamino, diethylamino, ethylamino, methylamino, and the like.

A “C-amido” group refers to a —C(═O)NR¹⁰R¹¹ group, with R¹⁰ and R¹¹ as defined herein. For example, R¹⁰ is hydrogen or unsubstituted C₁-C₄ alkyl and R¹¹ is hydrogen, C₁-C₄ alkyl optionally substituted with heteroalicyclic, hydroxy, or amino. For example, C(═O)NR¹⁰R¹¹ may be aminocarbonyl, dimethylaminocarbonyl, diethylaminocarbonyl, diethylaminoethylaminocarbonyl, ethylaminoethylaminocarbonyl, and the like.

An “N-amido” group refers to a R¹¹C(═O)NR¹⁰— group, with R¹⁰ and R¹¹ as defined herein, e.g. acetylamino, and the like.

“C-carboxy” and “carboxy” which are used interchangeably herein refer to a —C(═O)O—R″ group, with R″ as defined herein, e. g. —COOH, methoxycarbonyl, ethoxycarbonyl, benzyloxycarbonyl, and the like.

An “O-carboxy” group refers to a —OC(═O)R″ group, with R″ as defined herein, e.g. methylcarbonyloxy, phenylcarbonyloxy, benzylcarbonyloxy, and the like.

A “nitro” group refers to a —NO₂ group.

A “sulfinyl” group refers to a —S(O)—R″ group, wherein, R″ is selected from the group consisting of hydrogen, hydroxy, alkyl, cycloalkyl, aryl, heteroaryl (bonded through a ring carbon) and heteroalicyclic (bonded through a ring carbon), as defined herein.

A “sulfonyl” group refers to a —S(O)₂R″ group wherein, R″ is selected from the group consisting of hydrogen, hydroxy, alkyl, cycloalkyl, aryl, heteroaryl (bonded through a ring carbon) and heteroalicyclic (bonded through a ring carbon), as defined herein.

The compounds of and used in the invention are inclusive of all possible stereo-isomers of the respective compounds, including tautomers, geometric isomers, e.g. Z and E isomers (cis and trans isomers), and optical isomers, e.g. diastereomers and enantiomers. Furthermore, the invention includes in its scope both the individual isomers and any mixtures thereof, e.g. racemic mixtures. In this context, the term “isomer” is meant to encompass all optical isomers of the compounds described herein. It will be appreciated by those skilled in the art that the compounds described herein may contain at least one chiral center. Accordingly, the compounds of the invention may exist in optically active or racemic forms. It is to be understood that the compounds according to the present invention may encompass any racemic or optically active form, or mixtures thereof. In one embodiment, the compounds of the invention can be pure (R)-isomers. In another embodiment, the compounds of the invention can be pure (S)-isomers. In another embodiment, the compounds of the invention can be a mixture of the (R) and the (S) isomers. In a further embodiment, the compounds of the invention can be a racemic mixture comprising an equal amount of the (R) and the (S) isomers. The individual isomers may be obtained using the corresponding isomeric forms of the starting material or they may be separated after the preparation of the end compound according to conventional separation methods. For the separation of optical isomers, e.g. enantiomers, from the mixture thereof, the conventional resolution methods for example fractional crystallization may be used.

A “pharmaceutical composition” refers to a mixture of one or more of the compounds described herein, or physiologically/pharmaceutically acceptable salts or prodrugs thereof, with other chemical components, such as physiologically/pharmaceutically acceptable carriers and excipients. The purpose of a pharmaceutical composition is to facilitate administration of a compound to a subject.

As used herein, the term “pharmaceutically acceptable salt” refers to those salts which retain the biological effectiveness and properties of the parent compound. Such salts include, but are not restricted to: (1) an acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid; or (2) salts formed when an acidic proton present in the parent compound either is replaced by a metal ion, e. g., an alkali metal ion, such as sodium or potassium, an alkaline earth ion, such as magnesium or calcium, or an aluminum ion; or coordinates with an organic base such as ethanolamine, diethanolamine, triethanolamine, tromethamine, N-methylglucamine, and the like.

The compound of Formula I may also act as a prodrug. A “prodrug” refers to an agent which is converted into the parent drug in vivo. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but then is metabolically hydrolyzed to the carboxylic acid, the active entity, once inside the cell where water solubility is beneficial. A prodrug may be converted into the parent drug by various mechanisms, including enzymatic processes and metabolic hydrolysis.

A further example of a prodrug might be a short polypeptide, for example, without limitation, a 2-10 amino acid polypeptide, bonded through a terminal amino group to a carboxy group of a compound of this invention wherein the polypeptide is hydrolyzed or metabolized in vivo to release the active molecule. The prodrugs of compounds of Formula I are within the scope of this invention.

As used herein, a “physiologically/pharmaceutically acceptable carrier” refers to a carrier or diluent that does not cause significant irritation to an organism or subject and does not abrogate the biological activity and properties of the administered compound.

A “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatine, vegetable oils and polyethylene glycols.

The term “antibiotic” or “anti-bacterial agent” relates to a compound that inhibits, abrogates or prevents the growth of microbes, such as bacteria.

“Bacterial infection” relates to an infection of an organism with microbes or bacteria, for example pathogenic bacteria. The bacteria may, for example, be selected from the genus Actinobacteria, Actinomyces, Bacillus, Clostridium, Corynebacterium, Enterococcus, Vancomycin-resistant Enterococcus (VRE), Lactobacillales, Listeria, Mycobacterium, Norcardia, Propionibacterium, Rhodococcus, Sarcina, Solobacterium, Staphylococcus or Streptococcus.

“Treat”, “treating” and “treatment” refer to a method of alleviating or abrogating a bacterial infection and/or its attendant symptoms.

“Prevent”, “preventing” and “prevention” refer to a method of hindering a bacterial infection from occurring, i.e. a prophylactic method.

“Subject” refers to a living organism, for example a mammal, including a human being.

“Therapeutically effective amount” refers to that amount of the compound being administered which will relieve to some extent one or more of the symptoms of the disease/infection being treated.

### Illustrative Embodiments

The present invention is based on the inventor's surprising finding that the compounds of the present invention have significant properties in fluorescent imaging and photodynamic inactivation of bacteria, in particularly bacteria resistant or sensitive to conventional antibiotics such as vancomycin.

### Compounds

The compounds are typically compounds of formula (I)

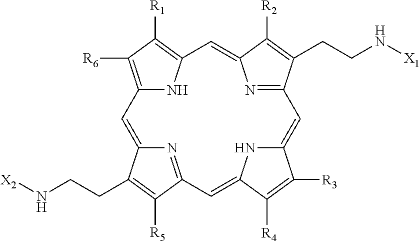

wherein

R₁ to R₆ are each independently selected from the group consisting of unsubstituted or substituted C₁-C₁₀ alkyl, unsubstituted or substituted C₁-C₁₀ alkenyl, unsubstituted or substituted C₁-C₁₀ alkynyl and unsubstituted or substituted C₁-C₁₀ alkoxy;

X₁ and X₂ are each independently H or vancomycin;

or a tautomer, stereoisomer, pharmaceutically acceptable salt or prodrug thereof.

In various embodiments of the present invention, R₁ to R₆ are each independently unsubstituted or substituted C₁-C₁₀ alkyl. In other embodiments, R₁ to R₆ are each independently C₁-0₅ alkyl. In specific embodiments, each of R₂, R₃, R₅ and R₆ is a methyl. In other specific embodiments, each of R₁ and R₄ is a pentyl or n-pentyl.

In various embodiments, the compound of the present invention has formula II or Formula III

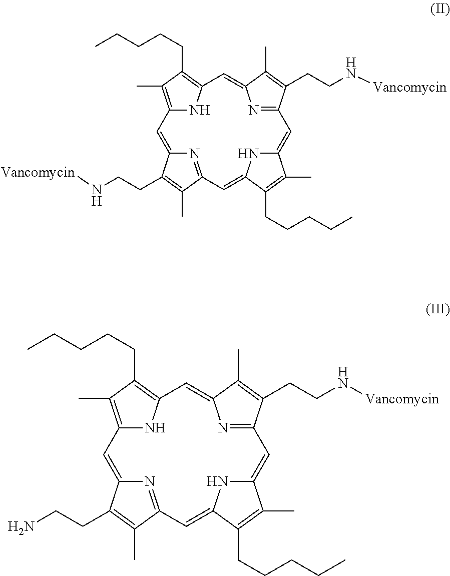

### Utility

In some embodiments, the compounds of the invention are used in a method for the treatment of prevention of a bacterial infection in a subject or organism. The bacterial infection may be caused by a Gram positive bacterium. The bacterial infection may, for example, be caused by bacteria of the genus Actinobacteria, Actinomyces, Bacillus, Clostridium, Corynebacterium, Enterococcus, Vancomycin-resistant Enterococcus (VRE), Lactobacillales, Listeria, Mycobacterium, Norcardia, Propionibacterium, Rhodococcus, Sarcina, Solobacterium, Staphylococcus or Streptococcus. In one particular embodiment, the infection is caused by Actinomyces israelii, Actinomyces naeslundii, Bacillus subtilis, Bacillus anthracis, Bacillus cereus, Clostridium botulinum, Clostridium difficile, Clostridium perfringens, Clostridium tetani, Clostridium sordellii, Corynebacterium diphtheriae, Corynebacterium jeikeium, Corynebacterium minutissimum, Enterococcus avium, Enterococcus durans, Enterococcus faecalis, Enterococcus faecium, Enterococcus gallinarum, Enterococcus solitarius, Listeria monocytogenes, Nocardia asteroids, Nocardia brasiliensis, Propionibacterium acnes, Rhodococcus equi, Sarcina ventriculi, Solobacterium moorei or Staphylococcus aureus. The subject affected by the bacterial infection may be a mammal, such as a human being.

In another embodiment of the invention, the compounds of the invention are suitable for use as a photodynamic antimicrobial (antibacterial) agent. In some embodiments, the compounds of the invention are suitable for killing bacteria which have developed resistance to conventional antibiotic treatments such as vancomycin resistant Enterococcus. When a compound of the invention is used as a photodynamic antibacterial agent to treat a subject, the compound is capable of emitting reactive oxygen species or oxygen free radicals following illumination irradiation with a light source of an appropriate wavelength. The wavelength is typically in the range of about 400 nm to about 800 nm; or about 400 nm to about 700 nm; or about 400 nm to about 600 nm; or about 400 nm to about 500 nm; or about 450 nm; or about 500 nm; or about 550 nm; or about 600 nm. In other embodiments, the light source emits light at fluence in the range of about 0 to about 60 J/cm²; or about 0 to about 50 J/cm²; or about 0 to about 40 J/cm²; or about 0 to about 30 J/cm²; or about 0 to about 20 J/cm².

When a light source is used in some embodiments of the invention, the light source can be a laser light source, a high intensity flash lamp, an emitting diode LED or other illumination source as by those skilled in the relevant arts. A broad light source may be utilized although a narrow spectrum source may be a preferred light source. In some embodiments, the light source can be emitted for a period of time necessary to effect a response. The period of time for illumination may be between 5 and 1 hour, or more preferably the period of time for illumination may be between 2 and 20 minutes.

It will be appreciated by persons skilled in the art that the compounds of the invention are suitable to treat all microbial infections regardless of whether the site of infection is light accessible or not. Thus, the compounds may have utility to treat infections which are not able to be treated by conventional photodynamic therapy agents. In some embodiments, the compounds of the invention can be used to treat infections where target microorganisms can be found on a light accessible surface or in a light accessible area, for example, epidermis, oral cavity, nasal cavity, sinuses, ears, eyes, lungs, urogenital tract and gastrointestinal tract. In addition, the compounds of the invention may be suitable to treat infections on surfaces or areas which are made accessible to light transiently such as infected bones temporarily exposed during surgical procedures. Infections of the peritoneal cavity such as those resulting from burst appendicitis are light accessible via at least laparoscopic devices.

The compounds of the invention are also found to be useful as fluorescence probes for imaging bacteria, in particularly live bacterial strains. Thus, the invention provides a method of detecting a bacterium. The method includes contacting said bacterium with at least one compound of the present invention. The bacterium can, for example, be detected by detecting the binding between the compound and the said bacterium via fluorescent imaging. In some embodiments, the method can be carried out in vivo or in vitro. The compounds of the invention demonstrate high binding affinity to bacteria by targeting the bacterial cell wall. Upon the specific targeting of the vancomycin moiety of the compound to the bacteria, the compound of the invention emits fluorescent signals, thereby detecting the bacteria. A stronger fluorescence would indicate a higher binding association of the compound with the bacteria.

Any bacteria can be used so long as the at least one compound of the invention is capable of targeting the respective bacterium. The bacterium can, in some embodiments, be a Gram positive bacterium including any of the Gram positive bacterium described herein. In a specific embodiment, the Gram positive bacterium is a Vancomycin-resistant Enterococcus (VRE).

### Pharmaceutical Compositions and Administration Thereof

A compound of the present invention or a pharmaceutically acceptable salt thereof, can be administered as such to a human patient or can be administered in pharmaceutical compositions in which the foregoing materials are mixed with suitable carriers or excipient(s). Techniques for formulation and administration of drugs are within the knowledge of persons skilled in the art. As used herein, “administer” or “administration” refers to the delivery of a compound of Formula (I) or a pharmaceutically acceptable salt thereof or of a pharmaceutical composition containing a compound of Formula (I) or a pharmaceutically acceptable salt thereof of this invention to an organism for the purpose of prevention or treatment of a bacterial infection.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. The preferred routes of administration are oral and parenteral.

Alternatively, one may administer the compound in a local rather than systemic manner, for example, via injection of the compound directly into a vessel, optionally in a depot or sustained release formulation.

Pharmaceutical compositions of the present invention may be manufactured by processes well known in the art, e. g., by means of conventional mixing, dissolving, granulating, drageemaking, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatine, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatine, as well as soft, sealed capsules made of gelatine and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also.

The compounds may also be formulated for parenteral administration, e. g., by bolus injection or continuous infusion. Formulations for injection may be presented in unit dosage form, e. g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound.

Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextrane. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e. g., sterile, pyrogen-free water, before use.

The compounds may also be formulated in rectal compositions such as suppositories or retention enemas, using, e. g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

A non-limiting example of a pharmaceutical carrier for the hydrophobic compounds of the invention is a cosolvent system comprising benzyl alcohol, a nonpolar surfactant, a water-miscible organic polymer and an aqueous phase such as the VPD co-solvent system. VPD is a solution of 3% w/v benzyl alcohol,8% w/v of the nonpolar surfactant Polysorbate 80, and 65% w/v polyethylene glycol 300, made up to volume in absolute ethanol. The VPD co-solvent system (VPD: D5W) consists of VPD diluted 1:1 with a 5% dextrose in water solution. This cosolvent system dissolves hydrophobic compounds well, and itself produces low toxicity upon systemic administration.

Naturally, the proportions of such a co-solvent system may be varied considerably without destroying its solubility and toxicity characteristics. Furthermore, the identity of the co-solvent components may be varied: for example, other low toxicity nonpolar surfactants may be used instead of Polysorbate 80, the fraction size of polyethylene glycol may be varied, other biocompatible polymers may replace polyethylene glycol, e.g., polyvinyl pyrrolidone, and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semipermeable matrices of solid hydrophobic polymers containing the therapeutic agent.

Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for stabilization may be employed.

The pharmaceutical compositions herein also may comprise suitable solid or gel phase carriers or excipients. Examples of such carriers or excipients include, but are not limited to, calcium carbonate, calcium phosphate, various sugars, starch, cellulose derivatives, gelatine, and polymers such as polyethylene glycols.

Many of the compounds of the invention may be provided as physiologically acceptable salts wherein the claimed compound may form the negatively or the positively charged species. Examples of salts in which the compound forms the positively charged moiety include, without limitation, the sodium, potassium, calcium and magnesium salts formed by the reaction of a carboxylic acid or sulfonic acid group in the compound with an appropriate base (e.g. sodium hydroxide (NaOH), potassium hydroxide (KOH), Calcium hydroxide (Ca(OH)₂), etc.).

Pharmaceutical compositions suitable for use in the present invention include compositions wherein the active ingredients are contained in an amount sufficient to achieve the intended purpose, e. g., the treatment of a bacterial infection.

More specifically, a therapeutically effective amount means an amount of compound effective to prevent, alleviate or ameliorate symptoms of bacterial infection or prolong the survival of the subject being treated.

Determination of a therapeutically effective amount is well within the capability of those skilled in the art, especially in light of the detailed disclosure provided herein.

For any compound used in the methods of the invention, the therapeutically effective amount or dose can be estimated initially from the described assays. Then, the dosage can be formulated for use in animal models so as to achieve a circulating concentration range that includes the MIC as determined in the experiments (i.e., the minimum concentration of the test compound which achieves inhibition of bacterial growth). Such information can then be used to more accurately determine useful doses in humans.

Toxicity and therapeutic efficacy of the compounds described herein can be determined by standard pharmaceutical procedures in cell cultures or experimental animals, e.g., by determining the MIC and the LD₅₀ for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition.

Dosage amount and interval may be adjusted individually to provide plasma levels of the active species which are sufficient to maintain the anti-bacterial effect. These plasma levels are referred to as minimal effective concentrations (MECs).

Dosages necessary to achieve the MEC will depend on individual characteristics and route of administration. HPLC assays or bioassays can be used to determine plasma concentrations.

Dosage intervals can also be determined using MEC value.

Compounds should be administered using a regimen that maintains plasma levels above the MEC for 10-90% of the time, preferably between 30-90% and most preferably between 50-90%.

In cases of local administration or selective uptake, the effective local concentration of the drug may not be related to plasma concentration and other procedures known in the art may be employed to determine the correct dosage amount and interval.

The amount of a composition administered may, of course, be dependent on the subject being treated, the severity of the affliction, the manner of administration, the judgment of the prescribing physician, etc.

The compositions may, if desired, be presented in a pack or dispenser device, such as a kit approved by a regulatory authority, such as EMEA or FDA, which may contain one or more unit dosage forms containing the active ingredient. The pack may for example comprise metal or plastic foil, such as a blister pack. The pack or dispenser device may be accompanied by instructions for administration.

The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration.

Compositions comprising a compound of the invention formulated in a compatible pharmaceutical carrier may also be prepared, placed in an appropriate container, and labelled for treatment of an indicated condition.

### Synthesis

The invention also provides a method of preparing a compound of formula (I). The method includes reacting vancomycin hydrochloride with a compound of formula (IV)

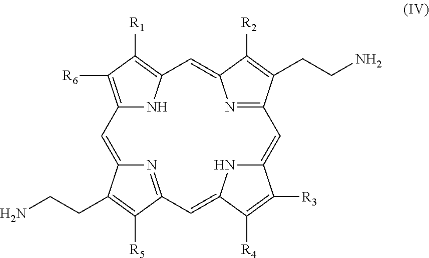

in the presence of a coupling reagent, under conditions to form a compound of formula (I), wherein
R₁ to R₆ are each independently selected from the group consisting of unsubstituted or substituted C₁-C₁₀ alkyl, unsubstituted or substituted C₁-C₁₀ alkenyl, unsubstituted or substituted C₁-C₁₀ alkynyl and unsubstituted or substituted C₁-C₁₀ alkoxy. In some embodiments, the coupling agent used in the method of the invention is O-benzotriazol-1-yl-N,N,N′,N′-tetramethyl-uronium-hexafluorophosphate (HBTU).

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of

Markush groups, those skilled in the art will recognize that the invention is also thereby described in terms of any individual member or subgroup of members of the Markush group.

### EXAMPLES

### General

Chemical reagents and solvents were used as received from commercial sources unless otherwise stated. UV-vis spectra were recorded in a 5-mm path quartz cell on a Beckman coulter DU800 spectrometer. Fluorescence spectroscopic studies were carried out on Varian Cary Eclipse Fluorescent Spectrometer. Fluorescence imaging was acquired with a confocal fluorescence microscope (Nikon Eclipse TE2000-E), using a super high pressure mercury lamp (Nikon, TE2-PS100W) with an excitation filter: 535/50 nm and emission filter: 610/75 nm. High performance liquid chromatography (HPLC) was performed on a reverse-phase column with a Shimadzu HPLC system. Analytical reverse-phase high performance liquid chromatography (RP-HPLC) was performed on Alltima C-18 column (250×3.0 mm) at a flow rate of 1.0 mL/min and semi-preparative RP-HPLC was performed on the similar C-18 column (250×10 mm) at a flow rate of 3 mL/min. HPLC elution employed linear gradients of [0.1% trifluoroacetic acid (TFA) in water (solution A)] and [0.1% TFA in acetonitrile (solution B)]. The linear gradient started from 70% solution A and 30% solution B, changed to 68.5% solution A and 31.5% solution B in 18 min, and to 0% solution A and 100% solution B in the following 12 min, and then to 70% solution A and 30% solution B in the next 5 min. 1H NMR spectra were obtained on a 300 MHz Bruker Advance in DMSO-d6. MALDI-MS spectrometric analyses were performed at the Mass Spectrometry Facility of School of Biological Sciences, Nanyang Technological University, Singapore.

### Example 1

Synthesis of monovalent Van-porphyrin 3a (compound of formula III): Vancomycin hydrochloride (52.8 mg, 35.9 μmol, 1.04 equiv.) and porphyrin 2 (20.8 mg, 35.1 μmol, 1.00 equiv.)^{[15]} were dissolved in 2 mL of dry dimethyl sulfoxide (DMSO). The mixture was cooled to 0° C., and O-benzotriazol-1-yl-N,N,N′,N′ tetramethyluronium hexafluorophosphate (HBTU) (38.0 mg, 100.2 μmol, 2.85 equiv.) in 1 mL of dry N,N-dimethylformamide (DMF) was added, followed by N,N-diisopropylethylamine (DIEA) (0.06 mL, 344 μmol, 9.8 equiv.). The mixture was allowed to rise to room temperature and stirred overnight. The reaction was quenched by adding dropwise 20 mL of acetone. A deep purple solid was precipitated, filtered out and was washed once by 5 mL of acetone. The crude product was purified by reversed-phase HPLC (RP-HPLC) to give 37.4 mg (18.5 μmol) of pure product (yield: 52.9%). ¹H-NMR (300 MHz, DMSO-d6): 10.28 (br s), 10.22 (s), 10.27 (s), 9.43 (br s), 9.12(br s), 8.68 (br s), 8.60 (br s), 8.18 (s), 7.97 (s), 7.79 (d, 8.6 Hz), 7.50-7.55 (overlapped), 7.28-7.40 (overlapped), 6.82 (s), 6.62 (s), 6.45 (s), 6.09 (br s), 5.81 (br s), 5.57 (s), 5.39 (s), 5.27 (s), 5.18 (br s), 4.90 (br s), 4.70 (d, 5.7Hz), 4.30-4.38 (overlapped), 4.03-4.14 (overlapped), 3.70 (m), 2.88 (s), 2.69 (s), 2.38 (s), 2.26 (m), 1.91 (s), 1.70 (m), 1.54 (m), 1.35 (s), 1.10 (m), 0.85-0.95 (overlapped). MALDI-ToF-MS: The peaks at m/z 2025 correspond to M⁺, respectively.

### Example 2

Synthesis of divalent Van-porphyrin 3b (compound of formula II): Vancomycin hydrochloride (106.5 mg, 71.7 μmol, 2.04 equiv.) and 2 (20.8 mg, 35.1 μmol, 1.00 equiv.) were dissolved in 2 mL of dry dimethyl sulfoxide (DMSO). The mixture was cooled to 0° C., and O-benzotriazol-1-yl-N,N,N,N′ tetramethyluronium hexafluorophosphate (HBTU) (38.0 mg, 100.2 μmol, 2.85 equiv.) in 1 mL of dry N,N-dimethylformamide (DMF) was added, followed by N,N-diisopropylethylamine (DIEA) (0.06 mL, 344 μmol, 9.8 equiv.). The mixture was allowed to rise to room temperature and stirred overnight. The reaction was quenched by adding dropwise 20 mL of acetone. A deep purple solid was precipitated, filtered out and was washed once by 5 mL of acetone. The crude product was purified by reversed-phase HPLC (RP-HPLC) to give 64.9 mg (18.8 μmol) of pure product (yield: 53.6%). ¹H-NMR (300 MHz, DMSO-d6): 8.98 (br s), 8.69 (br s), 7.94 (s), 7.67 (br s), 7.54 (br, s), 7.28 (d, 8.4 Hz), 6.92 (d, 8.0 Hz), 6.80 (d, 8.1 Hz), 6.64 (br s), 6.54 (s), 6.43 (s), 6.37 (s), 5.96 (br s), 5.78 (br s), 5.62 (br s), 5.39 (br s), 5.23 (br s), 5.18 (br s), 4.93 (br m), 4.66 (d, 5.7 Hz), 4.23 (overlapped), 4.06 (br, m), 3.80 (br m), 3.69 (overlapped), 3.27 (br s), 3.04-2.88 (multiple), 2.63 (s), 2.28-2.10 (overlapped), 1.91-1.69 (overlapped), 1.61-1.56 (multiple), 1.30 (s), 1.12 (t, 13.6 Hz), 0.94 (d, 6.4Hz), 0.88 (d, 5.5 Hz). MALDI-ToF-MS: calc. M⁺=3455.3, obsvd., the peaks at m/z 3455 correspond to M⁺, respectively.

### Example 3

In order to ascertain that the porphyrin is covalently bonded to Van, both 3a and 3b were effectively separated by reverse-phase HPLC and both molecules show characteristic retention times. On the other hand, a mixture consisting of porphyrin and Van shows only the retention times of the individual species. This result rules out the possibility of non-covalent adduct formation between Van and porphyrin. In addition, the MALDI-ToF-MS results clearly yield the precise mass of 3a and 3b, indicating the formation of covalently Van bonded Van and porphyrin moiety.

### Example 4

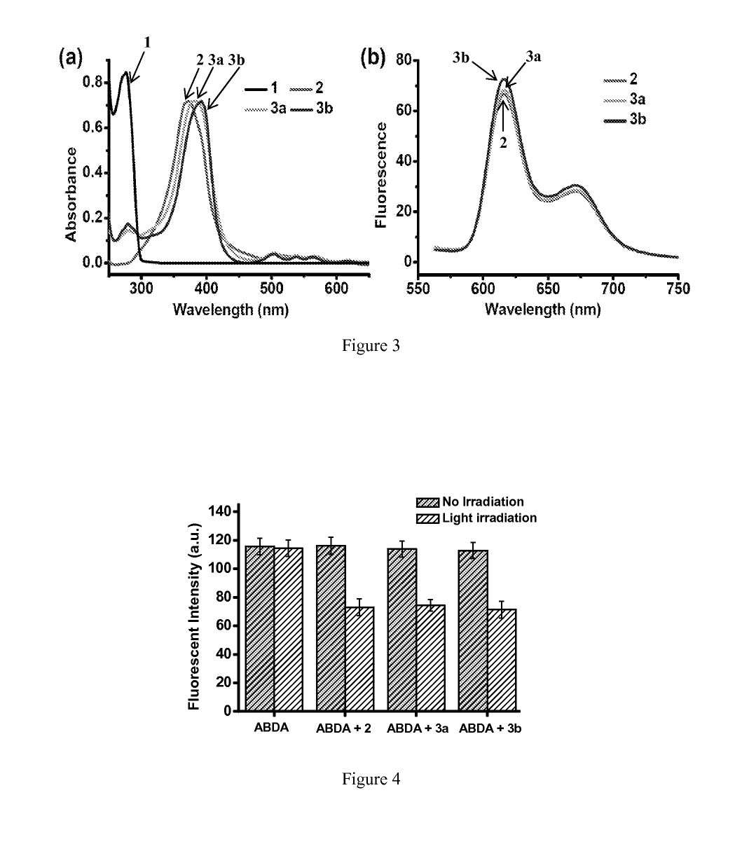

UV-Vis and Fluorescence Detection: 2 mM stock solution (in DMSO) of Porphyrin (2), monovalent (3a) and divalent (3b) Van-porphyrin were diluted to 20 μM in 10 mM PBS, pH 7.2, containing 1.0% DMSO as co-solvent. 200 μM vancomycin was also prepared in 10 mM PBS, pH=7.2. 600 μl solution of each compound was added into a 5-mm path quartz cell and the UV-Vis spectra were recorded using a Beckman coulter DU800 spectrometer. Fluorescence spectroscopic studies were also performed using a Varian Cary Eclipse Fluorescence Spectrophotometer (see FIG. 3).^{[16]}

After obtaining the Van-porphyrin derivatives (3a-b), their photochemical properties were investigated. The UV-Visible and fluorescence spectroscopy results indicated that the monovalent and divalent Van derivatives exhibited absorption bands of both Van (˜280 nm) and porphyrin moieties (B band around 400 nm, Q bands between 500 and 620 nm). The emission spectra of the Van-porphyrin precursors showed no difference from those of porphyrin molecule (FIG. 51, ESI†), suggesting Van conjugations have no effect on the fluorescent property of porphyrin. Moreover, all the precursors could produce singlet oxygen upon white light illumination.

### Example 5

Measurements of singlet oxygen (¹O₂) generation: Porphyrin (2), monovalent (3a) and divalent (3b) Van-porphyrin (10 μM) was mixed with 9, 10-anthracenediyl-bis(methylene)dimalonic acid (ABDA) (20 μM) in PBS buffer (10 mM, pH 7.2) and placed in quartz cuvette. The sample solutions were illuminated with white light (400 nm-800 nm) isolated from the emission of a Xenon lamp for 2 min and then the fluorescent emission of ABDA was measured at 431 nm when excited at 380 nm. The same sample solutions without light irradiation were used as control. The destruction of ABDA indicated the generation of singlet oxygen (see FIG. 4).^{[17]}

### Example 6

MIC test: A standard broth dilution method was used to determine the MICs.^{[18]} Porphyrin (2), Van-porphyrin monovalent (3a) and divalent (3b) were dissolved in DMSO to obtain 4 mg/ml stock solution and 4 mg/ml vancomycin/H₂O stock solution was also prepared. A total of 250 μl of LB solution was added to a series of sterile test tubes, with an additional 218 μl added to the first one. 32 μl of a 4 mg/ml compound stock solution was added to the first test tube, and a series of 2-fold dilutions were prepared by transferring 250 μl to successive tubes. A 5-ml culture of three bacterial strains, Bacillus subtilis (ATCC 33677) Enterococcus faecium (ATCC 51559, Van A) and Enterococcus faecalis (ATCC 51299, Van B), was grown to an OD₆₀₀ of 0.5 in LB medium. A 10 μl bacterial solution was added to each tube containing different concentration of compounds. The final concentration of bacterial strains was 10⁵ colony forming units (CFU) per ml. The compound-treated cultures were incubated at 37° C. for 24 h, and the OD₆₀₀ was measured. The reported MICs were the lowest concentrations of compounds that prevented cell growth. Each measurement was performed in duplicate.

The in vitro antibacterial activities of the compounds (e.g. Van derivatives) of the invention were first investigated by standard broth microdilution assays. In this example, three bacterial strains: Van-sensitive strain, Bacillus subtilis (ATCC 33677) and two Van resistant enterococci (VRE) including Enterococcus faecium (VanA genotype, ATCC 51559) and Enterococcus faecalis (VanB genotype, ATCC 51299) were chosen as model organisms. Both monovalent (3a) and divalent (3b) Van derivatives showed effective MIC activity against Van sensitive B. subtilis which was similar to the parent Van molecule (Table 1, ESIt).

TABLE 1
The antibacterial activities (MIC) of vancomcyin and vancomycin-
porphyrin conjugates.

| | | MIC | |
|---|---|---|---|
| Com- | B. subtilis | E. faecium | E. faecalis |
| pds | (sensitive) | (VanA) | (VanB) |
| 2 | 1 μM (1.5 μg/ml) | >88.3 μM (128 μg/ml) | 44.2 μM (64 μg/ml) |
| 3a | 3 μM (6 μg/ml) | >15.8 μM (32 μg/ml)^{a} | >15.8 μM (32 μg/ml)^{a} |
| 3b | 2 μM (7 μg/ml)) | >18.5 μM (64 μg/ml) | >18.5 μM (64 μg/ml) |

^{a}The MIC values of the substrate for bacterial strains VanA and VanB cannot be further determined due to the low solubility in LB medium.

### Example 7

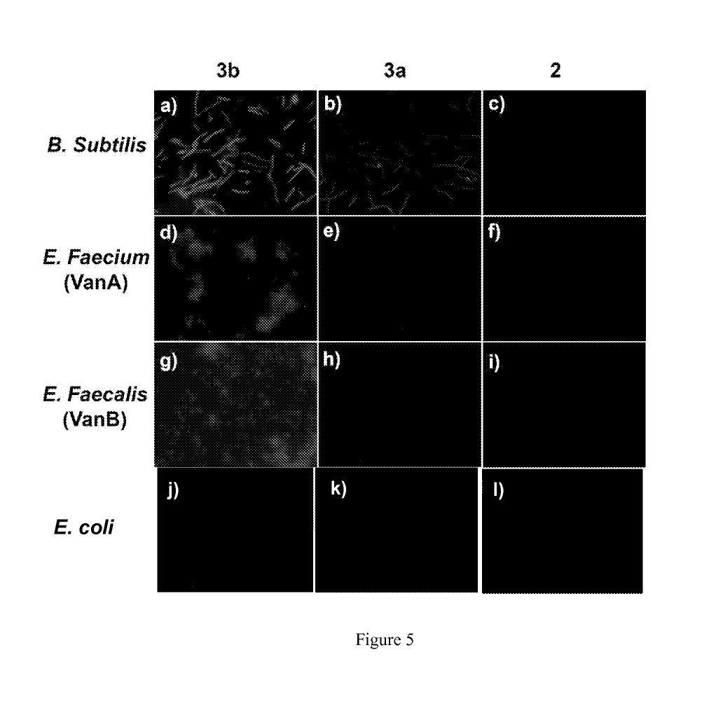

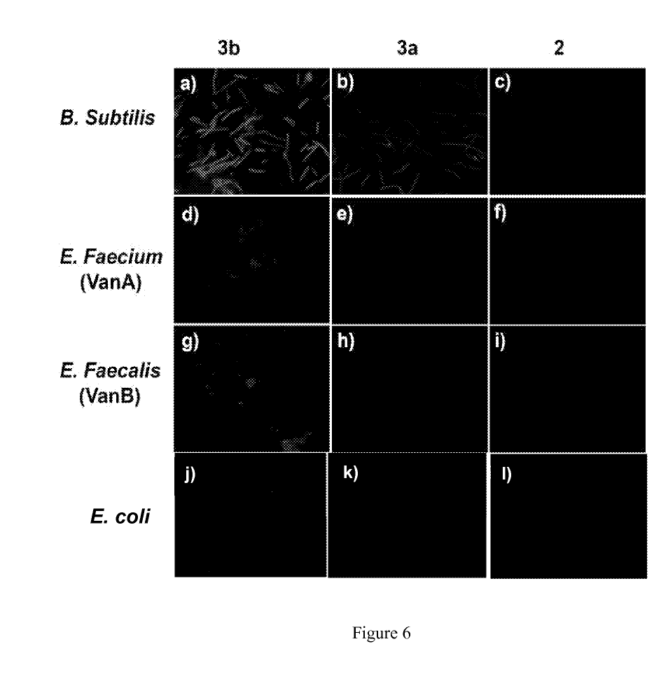

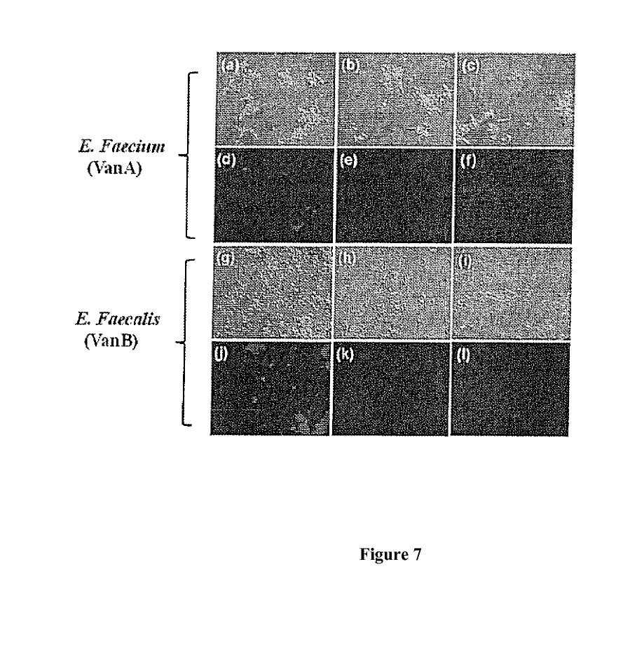

Imaging test: Single colonies of Escherichia coli (ATCC 53868), Bacillus subtilis (ATCC 33677) Enterococcus faecium (ATCC 51559, Van A) and Enterococcus faecalis (ATCC 51299, Van B) on solid Luria-Bertani (LB) plates were transferred to 5 ml of liquid LB culture medium and were grown at 37° C. for 12 h. Bacteria were harvested by centrifuging (4000 rpm for 10 min) and washed with sterile phosphate-buffered saline (PBS) three times. The supernatant was discarded and the remaining bacteria were re-suspended in PBS with an OD₆₀₀ of 0.5. Then, 2 μM or 10 μM of porphyrin (2), monovalent Van-porphyrin (3a) and divalent Van-porphyrin (3b) were added to bacterial cells suspensions and incubated in the dark for 1 hr at 37° C. After three times PBS washing, bacterial cells were spotted on glass slides and immobilized by the coverslips. Cell imaging tests were conducted with a Nikon Eclipse TE2000 Confocal Microscope. Images were captured with CFI VC 100 x oil immersed optics (see FIGS. 5 to 7).

The binding affinity of the compounds of the present invention towards various bacteria was further identified by fluorescent imaging technique. Typically, the bacterial strains were incubated with the compounds at 37° C. for 1 hour in culture media. After the cells were washed three times with PBS buffer to remove the unbound Van-porphyrins, the bacterial imaging was conducted upon the excitation of the Q bands of porphyrin under fluorescent microscope. As shown in FIG. 5c, incubation of porphyrin (2) itself with B. subtilis would not lead to obvious fluorescence. However, upon the specific targeting of Van affinity ligand, both 3a and 3b (2 μM) revealed obvious fluorescent signals in B. subtilis (FIGS. 5a and 5b). Without wishing to be bound by theory, compared to 3a, 3b exhibited stronger fluorescence, suggesting the higher binding association of 3b to the surface of B. subtilis. Similar bacterial imaging was also carried out by incubating VRE with 2 μM of 3a and 3b, separately. There was no significant fluorescence observed in these strains (FIG. 6) and the effective fluorescent imaging could only be detected when a higher concentration of 3b (10 μM) was used (FIGS. 5d and 5g), indicating the lower binding affinity of Van-porphyrins to the bacterial cell walls of VRE as compared to Van-sensitive bacterial strain. However, when compared to 3a, the multivalent/polyvalent interactions found in the divalent Van-porphyrin (3b) significantly improved the association between 3b and the drug resistance bacteria. In addition, FIG. 5 shows that incubation of 3b with VanA type VRE (FIG. 5d) displayed a lower fluorescent signal as compared to 3b incubated with VanB (FIG. 5g). This suggested a higher affinity between the divalent derivative and VanB strain. There was no obvious fluorescent signal observed in control E. coli imaging experiment indicating the lowest binding affinity between Van derivatives and Gram-negative strain.

### Example 8

Evaluation of photodynamic inactivation of bacterial strains: Photodynamic treatment was performed according to the methods previously described.^{[19]} Four bacterial strains, vancomycin-susceptible Gram-positive Bacillus subtilis (ATCC 33677), vancomycin-resistant Enterococcus faecium (ATCC 51559, Van A) and Enterococcus faecalis (ATCC 51299, Van B) and Gram-negative Escherichia coli (ATCC 53868), were used to evaluate the photodynamic killing of porphyrin (2), Van-porphyrin monovalent (3a) and divalent (3b). A single colony of bacteria was transferred to 5 ml of LB solution in the presence of 50 μg/ml ampicillin and was grown at 37° C. for 12 h. Then bacterial solutions were centrifuged at 4000 rpm for 10 min at 4° C. After washing with PBS three times, the bacteria were re-suspended in PBS with an OD₆₀₀ of 0.5. Then, cells were incubated with different concentrations of 2, 3a and 3b in the dark for 15 min at 37° C. All samples were illuminated with white light (400 nm-800 nm) isolated from the emission of a Xenon lamp. The time of illumination was adjusted from 0 to 2 min, corresponding to the total light doses of 0 to 100 J/cm². Following irradiation, bacterial suspensions were centrifuged (4000 rpm for 10 min, at 4° C.) and the supernatant was removed. After that, bacterial pellet was suspended and serially diluted (6×10⁴)-fold in PBS. A 100 μl portion of the diluted bacterial cells was spread on the solid LB agar plate and incubated for 16 hr at 37° C. The colonies formed were counted. The percentage of dead bacteria was evaluated by dividing the number of colony-forming units (cfu) between the samples incubated with the compounds of the invention and the control without the compounds and light exposure treatment.

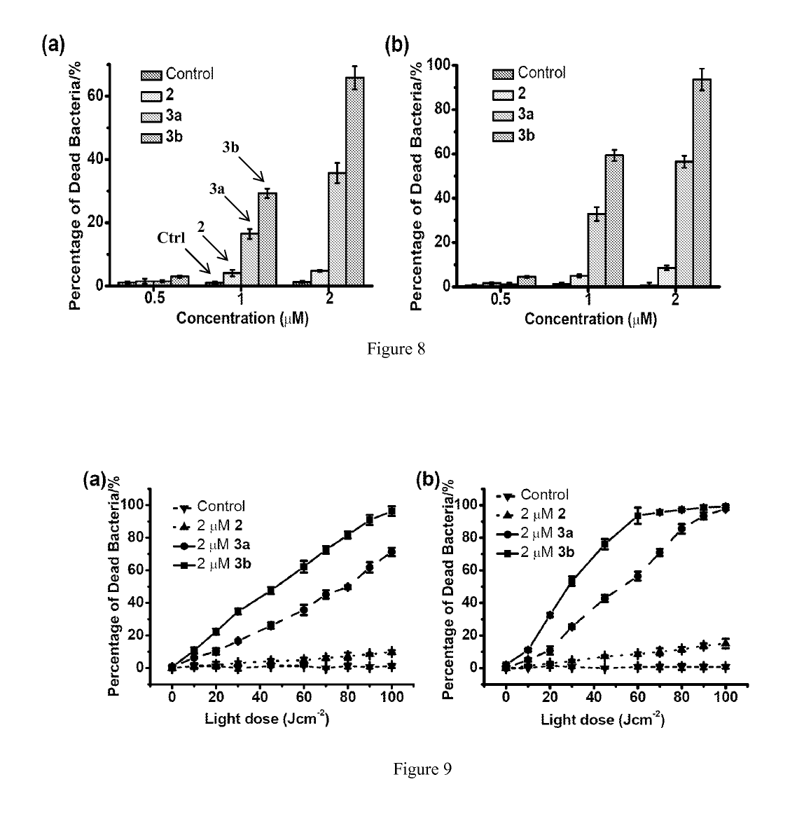

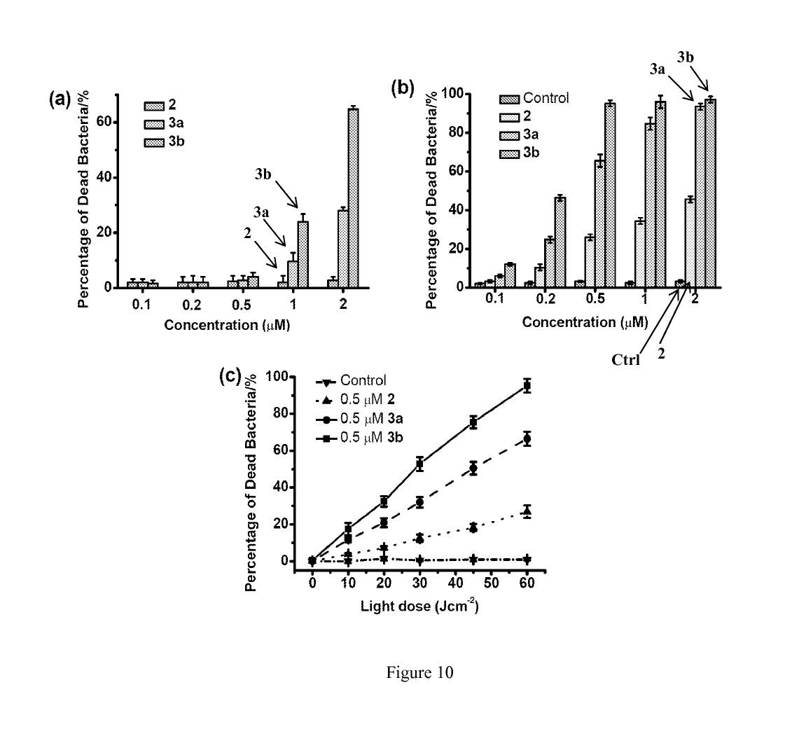

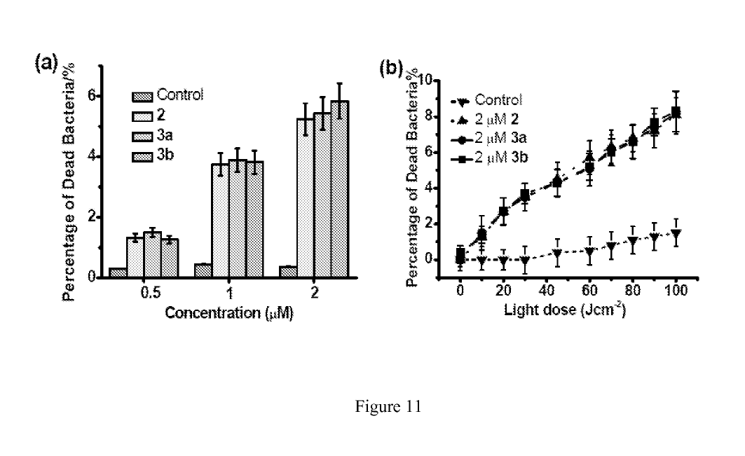

In order to further explore the photodynamic inactivation of VRE by the compounds of the invention, the PACT treatment was performed in the dark and upon white light exposure by a traditional surface plating approach. The compounds of the invention including 2, 3a and 3b were incubated with VanA and VanB, separately. Upon white light irradiation, the bacteria lethality was evaluated by counting the numbers of colony forming units (cfu) on LB agar plate. FIG. 8 displayed the bacterial lethality of VRE under different concentrations of the compounds. It was found that increasing concentrations of the compounds enhanced the bacterial killing efficiency for both VanA and VanB. Among the three compounds used, 3b showed the highest antibacterial activity against VRE throughout the whole concentration range. About 95% bacterial lethality could be observed in 3b (2 μM) incubated VanB upon irradiation with 60 J/cm² of white light, whereas, a smaller killing efficiency (˜66%) was detected for VanA suspension when exposed to the same dose of light. The photodynamic inactivation of both VanA and VanB were further investigated in the presence of different doses of white light (FIG. 9) while maintaining a fixed concentration (2 μM) of the invented compounds. Light irradiation of both VRE strains but no compounds incubation would not induce obvious bacterial damage which was used as the control. There was no significant bacterial lethality detected for 2 incubated VanA and VanB strains upon light exposure. On the other hand, 3a and 3b revealed the effective photodynamic inactivation of VanA and VanB upon exposing the bacteria to different doses of light and more significant bacterial reduction (e.g. >99%) could be achieved when higher doses of irradiation was applied (FIG. 9). Without wishing to be bound by theory, this showed that Van acted as an efficient affinity ligand and aided in targeting the porphyrin moiety to the VRE surfaces which resulted in an effective drug resistant bacterial lethality upon PACT treatment. Compared to 3a, 3b displayed substantially enhanced potency against VRE. The significant bacterial lethality achieved for VanA (˜66%) and VanB (˜95%) when 2 μM of 3b was incubated with VRE strains and irradiated with 60 J/cm² of white light was more potent than the MIC values of Van itself on VanA (˜44 times) and VanB (˜22 times) separately (Table 1). Moreover, the photodynamic inactivation was also carried out by incubating B. subtilis and E. coli with different concentrations of 2, 3a and 3b. Similarly, 3b displayed the highest potency against B. subtilis among the three compounds. More than 95% bacterial lethality was observed when 0.5 μM 3b incubated bacteria was exposed to 60 J/cm² of white light, which was more effective (˜4 times) than the value of 3b in MIC measurements (Table 1, FIG. 10). There was almost no lethality observed in E. coli for 2, 3a and 3b (FIG. 11). Without wishing to be bound by theory, these results unequivocally demonstrated that the compounds of the invention could serve as an effective photoactive antibacterial reagent against Van-sensitive and VRE strains due to the stronger association between 3b and the bacteria as a result of efficient multivalent/polyvalent interactions. This is consistent with the results observed in the bacterial imaging measurements.

In summary, the invented compounds exhibit a relatively higher binding affinity to bacterial surface and retain potent PACT activities against vancomycin-sensitive and VRE bacteria when compared to Van and porphyrin alone. Apart from the enhanced photodynamic antimicrobial activity, the red fluorescent emission of the compound can be used to carry out noninvasive imaging study in living bacterial strains.

### REFERENCES

1 (a) D. Kahne, C. Leimkuhler, W. Lu, C. T. Walsh, Chem. Rev. 2005, 105, 425. (b) L. H. Li, B. Xu, Curr. Pharm. Design. 2005, 11, 3111.
2 U. N. Sundram, J. H. Griffin, T. I. Nicas, J. Am. Chem. Soc. 1996, 118, 13107.
3 K. C. Nicolaou, R. Hughes, S. Y. Cho, N. Winssinger, C. Smethurst, H.Labischinski, R. Endermann, Angew. Chem. Int.Ed. 2000,39, 3823.
4 D. H. Williams, A. J. Maguire, W. Tsuzuki, M. S. Westwell, Science 1998, 280, 711.
5 (a) J. H. Rao, J. Lahiri, L. Isaacs, R. M. Weis, G. M. Whitesides, Science 1998, 280, 708. (b) J. H. Rao, J. Lahiri, R. M. Weis, G. M. Whitesides, J. Am. Chem. Soc. 2000, 122, 2698.
6 (a) B. G. Xing, C. W. Yu, P. L. Ho, K. H. Chow, T. Cheung, H. W. Gu, Z. W. Cai, B. Xu, J. Med. Chem. 2003, 46, 4904. (b) B. G. Xing, P. L. Ho, C. W. Yu, K. H. Chow, H. W. Gu, B. Xu, Chem. Comm. 2003, 2224. (c) H. Arimoto, K. Nishimura, T. Kinumi, I. Hayakawa, D. Uemura, Chem. Comm. 1999, 1361.
7 (a) R. K. Jain, J. Trias, J. A. Elllman, J. Am. Chem. Soc. 2003, 125, 8740. (b) P. J. Loll, P. H. Axelsen, Annu. Rev. Biophys. Biomol. Struct. 2000, 29, 265.
8 D. E. Dolmans, D. Fukumura, R. K. Jain, Nat. Rev. Cancer, 2003, 3, 380.
9 (a) J. P. Celli, B. Q. Spring, I. Rizvi, C. L. Evans, K. S. Samkoe, S. Verma, B. W. Pogue, T. Hasan. Chem. Rev. 2010,110, 2795. (b) A. P. Castano, P. Mroz, M. R. Hamblin, Nat. Rev. Cancer. 2006, 6, 535.
10 M. L. Embleton, S. P. Nair, B. D. Cookson, M. Wilson, J. Antimicrob. Chemother. 2002, 50, 857.
11 P. A. Suci, Z. Varpness, E. Gillitzer, T. Douglas, M. Young, Langmuir 2007, 23, 12280.
12. F. Gad, T. Zahra, K. P. Francis, T. Hasan, M. R. Hamblin, Photochem. Photobiol. Sci. 2004, 3, 451.
13 C. A. Strassert, M. Otter, R. Q. Albuquerque, A. Hone, Y. Vida, B. Maier, L. De Cola. Angew. Chem. Int. Ed. 2009, 48, 7928.
14 C. K. Hope, S. Packer, M. Wilson, S. P. Nair, J. Antimicrob. Chemother. 2009, 64, 5910.
15 B. Ward, P. M. Callahan, R. Young, G. T. Babcock, C. K. Chang, J. Am. Chem. Soc. 1983, 105, 634.
16 D. Dolphin, The porphyrin, 3, Physical chemistry, Part A.; Academic ress: New York, 1978, Vol. 3.
17 Qian, H. S.; Guo, H. C.; Ho, P. C. L.; Mahendran, R.; Zhang, Y. Small. 2009, 5, 2285-2290.
18 Washington, J. A. (1996) in Med. Microbiol., ed. Baron, S. (Univ. Texas Med. Branch, Galveston), p. 161
19 Xing C.; Xu. Q.; Tang H.; Liu L. and Wang S. J. Am. Chem. Soc.; 2009, 131, 13117-13124.

1. A compound of formula (I) 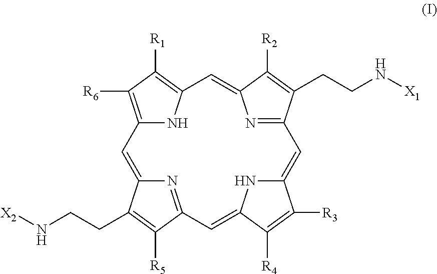
wherein
R₁ to R₆ are each independently selected from the group consisting of unsubstituted or substituted C₁-C₁₀ alkyl, unsubstituted or substituted C₁-C₁₀ alkenyl, unsubstituted or substituted C₁-C₁₀ alkynyl and unsubstituted or substituted C₁-C₁₀ alkoxy;
X₁ and X₂ are each independently H or vancomycin;
or a tautomer, stereoisomer, pharmaceutically acceptable salt or prodrug thereof.

2. The compound of claim 1, wherein R₁ to R₆ are each independently unsubstituted or substituted C₁-C₁₀ alkyl.

3. The compound of claim 1, wherein R₁ to R₆ are each independently C₁-C₅ alkyl.

4. The compound of claim 1, wherein each of R₂, R₃, R₅ and R₆ is a methyl.

5. The compound of claim 1, wherein each of R₁ and R₄ is a pentyl or n-pentyl.

6. The compound of claim 1, wherein the compound is a compound of Formula II or Formula III

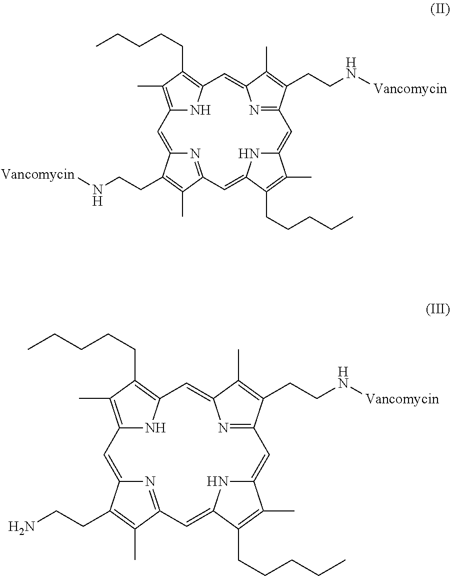

7. A composition comprising a compound according to claim 1.

8. The composition of claim 7, wherein the composition is a pharmaceutical composition.

9. The composition of claim 8, further comprising a pharmaceutically acceptable carrier.

10. A method of treating a bacterial infection in a subject comprising administering a therapeutically effective amount of the compound of claim 1 to a subject in need thereof.

11. The method of claim 10, wherein the bacterial infection is caused by a Gram positive bacterium.

12. The method of claim 10, wherein the bacterial infection is a Actinobacteria, Actinomyces, Bacillus, Clostridium, Corynebacterium, Enterococcus, Vancomycin-resistant Enterococcus (VRE), Lactobacillales, Listeria, Mycobacterium, Norcardia, Propionibacterium, Rhodococcus, Sarcina, Solobacterium, Staphylococcus or Streptococcus infection.

13. The method according to claim 12, wherein the bacterial infection is a Actinomyces israelii, Actinomyces naeslundii, Bacillus subtilis, Bacillus anthracis, Bacillus cereus, Clostridium botulinum, Clostridium difficile, Clostridium perfringens, Clostridium tetani, Clostridium sordellii, Corynebacterium diphtheriae, Corynebacterium jeikeium, Corynebacterium minutissimum, Enterococcus avium, Enterococcus durans, Enterococcus faecalis, Enterococcus faecium, Enterococcus gallinarum, Enterococcus solitarius, Listeria monocytogenes, Nocardia asteroids, Nocardia brasiliensis, Propionibacterium acnes, Rhodococcus equi, Sarcina ventriculi, Solobacterium moorei or Staphylococcus aureus infection.

14. The method of claim 10, wherein the subject is subjected to light irradiation with a light source.

15. The method of claim 14, wherein the light source emits a wavelength in the range of about 400 nm to about 800 nm.

16. The method of claim 14, wherein the light source emits light at fluence in the range of about 0 to about 60 J/cm².

17. A method of detecting a bacterium comprising contacting said bacterium with at least one compound of claim 1, wherein the bacterium is detected by detecting the binding between the compound and the said bacterium.

18. The method of claim 17, wherein the binding between the compound and the said bacterium is detected by fluorescent imaging.

19. The method of claim 17, wherein the method is an in vivo or an in vitro method.

20. The method of claim 17, wherein the bacterium is a Gram positive bacterium.

21. The method of claim 20, wherein the Gram positive bacterium is a Vancomycin-resistant Enterococcus (VRE).

22. A method of preparing a compound of formula (I) comprising reacting vancomycin hydrochloride with a compound of formula (IV) 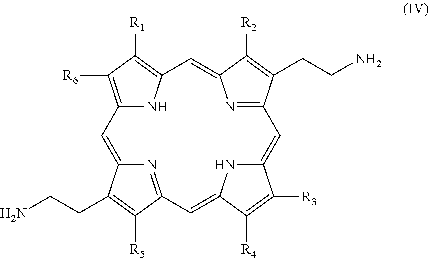
in the presence of a coupling reagent, under conditions to form a compound of formula (I), wherein
R₁ to R₆ are each independently selected from the group consisting of unsubstituted or substituted C₁-C₁₀ alkyl, unsubstituted or substituted C₁-C₁₀ alkenyl, unsubstituted or substituted C₁-C₁₀ alkynyl and unsubstituted or substituted C₁-C₁₀ alkoxy.

23. The method of claim 22 wherein R₁ to R₆ are each independently unsubstituted or substituted C₁-C₁₀ alkyl.

24. The method of claim 22, wherein R₁ to R₆ are each independently C₁-0₅ alkyl.

25. The method of claim 22, wherein each of R₂, R₃, R₅ and R₆ is a methyl.

26. The method of claim 22, wherein each of R₁ and R₄ is a pentyl or n-pentyl.

27. The method of claim 22, wherein the coupling agent is O-benzotriazol-1-yl-N,N,N′,N′-tetramethyl-uronium-hexafluorophosphate (HBTU).

28. A method of treating a bacterial infection in a subject comprising administering a therapeutically effective amount of the composition of claim 7 to a subject in need thereof.
